(19) United States
(12) Patent Application Publication
(10) Pub. No.: US 2019/0211024 A1
(43) Pub. Date: Jul. 11, 2019

(54) SMALL MOLECULES HAVING ANTIVIRAL PROPERTIES

(71) Applicants: Oleg I KISELEV, Saint-Petersburg (RU); Vladimir RUSINOV, Saint-Petersburg (RU); Evgeny ULOMSKY, Saint-Petersburg (RU); Natalia MEDVEDEVA, Saint-Petersburg (RU); Irina SAPOZHNIKOVA, Saint-Petersburg (RU); Valery CHARUSHIN, Saint-Petersburg (RU); Oleg CHUPAKHIN, Saint-Petersburg (RU); Daria DANILENKO, Saint-Petersburg (RU); Ella DEEVA, Saint-Petersburg (RU); GRL., INC., Gaithersburg, MD (US)

(72) Inventors: Oleg I Kiselev, Saint-Petersburg (RU); Evgeny Ulomsky, Saint-Petersburg (RU); Natalia Medvedeva, Saint-Petersburg (RU); Vladimir Rusinov, Saint-Petersburg (RU); Irina SAPOZHNIKOVA, Saint-Petersburg (RU); Valery CHARUSHIN, Saint-Petersburg (RU); Oleg CHUPAKHIN, Saint-Petersburg (RU); Daria Danilenko, Saint-Petersburg (RU); Ella DEEVA, Saint-Petersburg (RU)

(73) Assignee: General Research Laboratory, Gaithersburg, MD (US)

(21) Appl. No.: 16/326,253

(22) PCT Filed: Aug. 19, 2017
(86) PCT No.: PCT/US17/47699

§ 371 Date: Feb. 19, 2019

Related U.S. Application Data
(60) Provisional application No. 62/377,413, filed on Aug. 19, 2016.

(51) Int. Cl. C07D 487/04; A61P 31/12

(52) U.S. Cl. CPC C07D 487/04; A61P 31/12

(57) ABSTRACT

The present invention relates to salt forms and derivatives of 2-methylthio-6-nitro-1,2,4-triazoio[5,1-c]1,2,4-triazine -7-one, dehydrate and pharmaceutical compositions thereof. These salt forms and derivatives exhibit improved antiviral activity. The present invention also relates to processes for preparing the compounds, and intermediates used in their preparation.

### BACKGROUND OF THE INVENTION

There is a need for the development of new safe and effective antiviral compounds. According to statistics gathered by the World Health Organization (WHO), seasonal influenza (flu) occurs globally with an annual attack rate estimated at 5%-10% in adults and 20%-30% in children. Illnesses can result in hospitalization and death, especially among high-risk groups (the very young, elderly or chronically ill). Worldwide, these annual epidemics are estimated to result in about 3 to 5 million cases of severe illness, and about 250,000 to 500,000 deaths. Only two drugs, oseltamivir and zanamivir, are currently used for treatment of influenza and their effectiveness has been strongly challenged by the Cochrane foundation.

In addition to seasonal flu, pandemic flu can cause widespread morbidity and mortality. In 2009, a novel influenza A (H1N1) virus of swine origin caused human infection and acute respiratory illness globally, resulting in the first influenza pandemic since 1968 with circulation outside the usual influenza season in the Northern Hemisphere. The virus was a unique combination of influenza virus genes never previously identified in either animals or people. The virus genes were a combination of genes most closely related to North American swine-lineage H1N1 and Eurasian lineage swine-origin H1N1 influenza viruses. Such a reassortment was not anticipated by flu vaccine manufacturers and as a result, the global population had little or no immunity to the new virus.

By March 2010, almost all countries had reported cases, and more than 17,700 deaths among laboratory-confirmed cases had been reported to the World Health WHO. The number of laboratory-confirmed cases significantly underestimates the pandemic's impact. The CDC estimates the global death toll from the 2009 H1N1 influenza pandemic at more than 284,000, about 15 times the number of laboratory-confirmed cases. Importantly, only 20% of the deaths were in people older than 64 (in contrast, about 90% of seasonal flu deaths are in seniors).

The first avian influenza in humans was reported in Hong Kong in 1997. The outbreak was linked to chickens. Since then there have been human cases of avian influenza A (H5N1) in Asia, Africa, Europe, Indonesia, Vietnam, the Pacific, and the near East. Hundreds of people have become sick with this virus. Up to half of the people who get this virus die from the illness. The A(H5N1) and A(H7N9) Avian influenza viruses remain two of the influenza viruses with pandemic potential, because they continue to circulate widely in some poultry populations, most humans likely have no immunity to them, and they can cause severe disease and death in humans.

The SARS pandemic was due to a novel coronavirus that emerged in 2003 from China. The virus took the world by surprise as coronaviruses were not known to cause life-threatening pathologies. Coronaviruses were clearly neglected viruses from the scientific and the medical/veterinary point-of-view. According to WHO, a total of 8,098 people worldwide became sick with SARS during the 2003 outbreak. Of these, 774 died, mostly in the region around Hong Kong.

Respiratory syncytial virus, or RSV, is a respiratory virus that infects the lungs and breathing passages. Most otherwise healthy people recover from RSV infection in 1 to 2 weeks. However, infection can be severe in some people, such as certain infants, young children, and older adults. RSV is the most common cause of bronchiolitis and pneumonia in children under 1 year of age in the United States. In addition, RSV is more often being recognized as an important cause of respiratory illness in older adults. There is currently no vaccine available for humans and treatment primarily consists of intravenous infusions of immunoglobulin (palivizumab).

West Nile Virus (WNV) can cause neurological disease and death in people. WNV is commonly found in Africa, Europe, the Middle East, North America and West Asia. WNV is maintained in nature in a cycle involving transmission between birds and mosquitoes. Humans, horses and other mammals can be infected. Treatment is supportive for patients with neuro-invasive West Nile virus, often involving hospitalization, intravenous fluids, respiratory support, and prevention of secondary infections. No vaccine is available for humans and there are no approved drugs.

These and other viral outbreaks illustrate the need for effective antiviral agents. Vaccines, while effective, are not a complete solution. For example, for seasonal flu, only three viral strains typically are used for each year's seasonal vaccine and there is a mismatch between the strains chosen for vaccines and those that eventually become pandemic about 50% of the time. It takes 6-9 months each year to produce flu vaccines so new virulent strains aren't included. Even with a full match, those at highest risk (elderly and infants) do not respond well to vaccines. Finally, many people do not get even the approved vaccines.

The sodium salt of 2-methylthio-6-nitro-1,2,4-triazolo[5,1-c]1,2,4-triazine-7-one, dihydrate (Triazivirin or TZV) exhibits antiviral activity against influenza in both preclinical and clinical studies [Loginova et al., Antibiot Khimioter, 52(11-12):18-20, 2007; Lukovnikova et al. Med Tr Prom Ekol. (4):44-7, 2009; Loginova et al. Antibiot Khimioter. 55(9-10)25-8, 2010; Karpenko et al, Antimicrob Agents Chemother. 54(5):2017-22, 2010; Smirnova et al., Antibiot Khimioter. 56(11-12):11-6, 2011; Loginova et al., Antibiot Khimioter, 56(1-2):10-2, 2011; Loginova et al., Antibiot Khimioter. 57(11-12):8-10, 2012; Kiselev et al:, Vopr Virusol. November-December;57(6):9-12, 2012], RU 2294936 C1 discloses that the sodium salt of TZV has antiviral activity against influenza, Rift Valley fever virus, West Equine Encephalomyelitis (WEE) viruses, parainfluenza, respiratory-syncytium virus (RSV), Aujeszky's disease virus, avian infectious laryngotracheitis virus and avian influenza virus. RU 2444363 discloses the use of TZV for virus tick-borne encephalitis. RU 2343154 C2 discloses methods of synthesis of TZV. RU 2493158 C2 discloses a fluoro derivative of TZV. RU 2330036 discloses the synthesis of 5-methyl-6-nitro -1,2,4-triazolo [1,5-a] pyrimidin-7-one dehydrate. RU 2376307 discloses the synthesis of 4-((Z) 4 2-hydroxybutyl- [symbol] -Yl)-2-R-6-phenyl-1,2,4-triazolo [5,1-c] [1,2,4] triazin-7-ones and their activity on herpes simplex virus. RU 2340614 C2 discloses novel TZV derivatives (2-r-4-(allyloxymethyl)-6-nitro-1,2,4-triasolo[5,1-c]-1,2,4-triazin-7(4h)-ons and 24-r-4-(propargyloxymethyl)-6-nitro-1,2,4-triasolo[5,1-c]-1,2,4-triazin7(4h)-ons) with activity on influenza, RSV and herpes simplex viruses. RU 2455304 Cl discloses a novel TZV derivative (6-(2′-amino-2′-carboxyethylthio)-2-methylthio-4-(pivaloyloxy-methyl-1,2,4-triazolo(5, 1-c) 1,2,4-triazin-7(4H) and RU 2404182 C2 discloses a novel derivative (sodium salt of 2-ethylthio-6-nitro -1,2,4-triazole[5,1-c]-1,2,4-triazin-7-one dehydrate) of TZV. RU 2402552 C2 discloses the activity of novel TZV derivatives (sodium salt of 2-n-propylthio-6-nitro-1,2,4-triazole[5,1-c]-1,2,4-triazin-7(4H)-one dihydrate and a sodium salt of 2-n-butylthio-6-nitro-1,2,4-triazole[5,1-c]-1,2,4-triazin-7(4H)-one dehydrate) on herpes simplex virus and RU 2345080 C2 discloses the activity of a novel TZV derivative (4-(4′-hydroxybutyl)-6-phenyl-1,2,4-triazolo[5,1-c] [1,2,4]triazin-7-on) on herpes simplex virus. WO2013/122575 A2 discloses the TZV derivative 6-(2′-amino-2′-carboxyethylthio)-2-methylthio-4-pivaloyloxy-methyl-1,2,4-triazolo [5,1-c] 1,2,4 -triazin-7 (4H -one. Each reference and patent listed in this paragraph is incorporated by reference herein in its entirety.

The present invention relates to novel salt forms and derivatives of TZV with improved antiviral activity compared to the sodium salt of TZV and to pharmaceutical compositions thereof. These salt forms and derivatives exhibit improved antiviral activity. The present invention also relates to processes for preparing the compounds and intermediates used in their preparation.

### DETAILED DESCRIPTION

The present invention relates to salt forms and derivatives of Formula 1 (TZV).

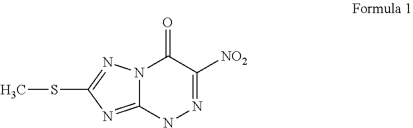

### Arginine Salt of TZV

In one embodiment, the salt form is the arginine salt of Formula 1. The arginine salt of Formula 1 surprisingly has increased antiviral activity compared to the sodium salt of Formula 1. Without being bound by any particular theory, the arginine salt of Formula 1 surprisingly has advantageous bioavailability when administered by the oral route, resulting in exceptionally high levels of the parent compound in the body. This enables less drug to be administered while still providing equivalent drug levels of the parent compound in the plasma. Oral administration with less dosage means patient compliance is considerably simplified.

The data below show that BSS14 (the aforementioned arginine salt of TZV) is comparable to Tamiflu in protecting mice from a lethal challenge from mice-adapted influenza infection and superior to the sodium salt of TZV. In these experiments, aliquots of the preparations were diluted in cell culture medium MEM Eagle (BioloT, St. Petersburg, cat. #1.3.3) and appropriately diluted in MEM for experiments on animals and in cell culture. As a reference drug, the sodium salt of triazavirin was used. A flu virus, A/California/07/09 (H1N1), that was adapted to mice was used in these studies. The virus was passaged in the allantoic cavity of chick embryos daily for 48 hours at 36° C. Albino mice (female) weighing 16-20 g were obtained from the kennel “Rappolovo” (Leningrad region) and kept on a standard diet in a regulated environment vivarium (Influenza Research Institute RAMS). Selection of animals in groups was conducted by random sampling. Prior to the test the animals were adapted to the animal facility for a week.

Homogenates of lung tissues of mice infected with a virus three days earlier were used for infection of test animals. From the homogenates was prepared a series of 10-fold dilutions in saline, after which the activity of infectious virus in the infecting material was determined in a separate experiment by titration of lethality in animals. The titer of virus was calculated by the method of Reed and Menchu (Am. J. Hyg. 27:493-497, 1938).

The test drugs were administered to animals orally by gavage in a volume of 0.2 mL (once daily for 5 days starting from the first days after infection of animals). The comparator drug was orally administered in the same way. The dose of drugs studied was 100 mg/kg body weight of animals per day.

The placebo control group was administered saline phosphate buffer. Untreated animals kept under the same conditions as the experimental group were used as a negative control.

The virus was administered intranasally to animals under light ether anesthesia using an LD50 dose. Each group consisted of 15 mice. The observation of the animals was performed for 14 days, i.e., the period during which the experimental animal influenza deaths were observed. Deaths of animals in the control and experimental groups were recorded daily. The mortality rate was calculated for each group (M, the ratio of dead animals for 14 days to the total number of infected animals per group), as were an index of protection (IP, odds ratio per cent mortality in the control and experimental groups to the percentage of mortality in the control group), and the average life span of animals (MDD) at the rate of 14 days of observation in accordance with the following formulas:

MDD=(ΣND)/Nt, where N=number of animals that lived and D=day Nt=total number of animals per group;
M=M/Nt, where M=number of animals in the group who died within 14 days after infection,
IP=((Mc−Me)/Mc)×100%, where Mc and Me=the percentage mortality in the control and experimental groups respectively.

In the course of the experiment to determine the protective activity of TZV analogs in mice there was no non-specific mortality observed in the control group of untreated animals.

Clinical symptoms were typical of influenza infection and included difficulty breathing, ataxia, tremor, and reduced feed intake and water, and as a consequence, the weight of the animals.

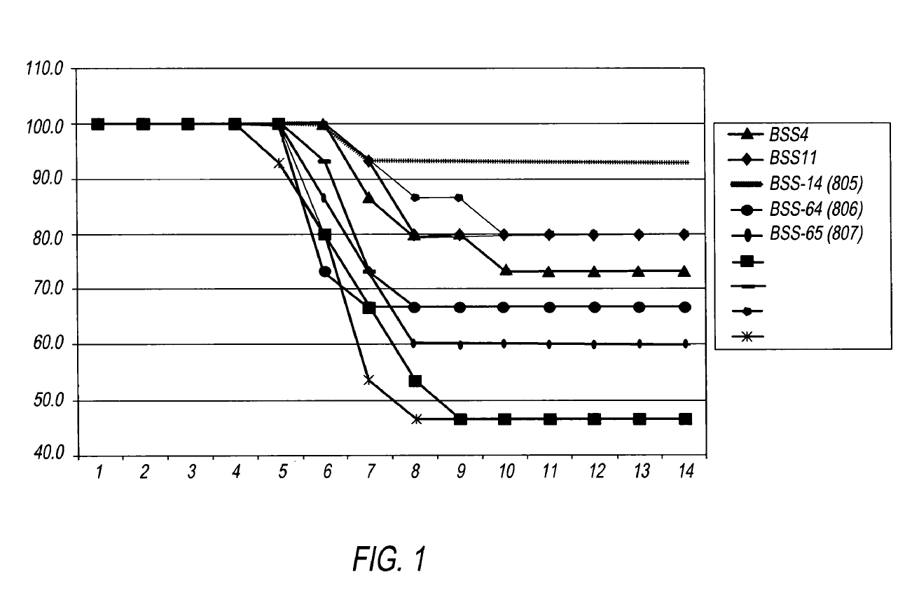

Data on the protective activity of the studied drugs are summarized in Table 1 and in FIG. 1.

TABLE 1
Activity of analogs against influenza virus A/California/07/09 (H1N1) pdm09

| | | | | | | | | | | | | Morta- | Index of | The |
|---|---|---|---|---|---|---|---|---|---|---|---|---|---|---|
| | Mortality by day | | | | | | | | | | AST | lity, | protection | increase |
| Preparation | 5 | 6 | 7 | 8 | 9 | 10 | 11 | 12 | 13 | 14 | cyr. | % | % | in AST, d. |
| BBS-64 (806) | | 4 | 1 | | | | | | | | 12.1 | 33.3 | 37.5 | 1.5 |
| BBS-65 (807) | | 2 | 2 | 2 | | | | | | | 11.8 | 40.0 | 25.0 | 1.3 |
| BBS-14 (805) | | | 1 | | | | | | | | 14.5 | 6.7 | 87.5 | 3.9 |
| mini 289 | | 1 | 3 | 1 | | | | | | | 12.3 | 33.3 | 37.5 | 1.8 |
| BSS11 | | | 1 | 2 | | | | | | | 13.5 | 20.0 | 62.5 | 3.0 |
| UPI-241 | | 3 | 2 | 2 | 1 | | | | | | 10.8 | 53.3 | 0.0 | 0.3 |
| BSS4 | | | 2 | 1 | | 1 | | | | | 13.1 | 26.7 | 50.0 | 2.6 |
| Triazavirin | | | 1 | 1 | | 1 | | | | | 13.7 | 20.0 | 62.5 | 3.1 |
| The Control | 1 | 2 | 4 | 1 | | | | | | | 10.5 | 53.3 | — | 0.0 |
| Virus | | | | | | | | | | | | | | |

FIG. 1 shows activity of analogs against influenza virus A/California/07/09 (H1N1) pdm09. As can be inferred from the data presented, influenza virus adapted to mice caused lethal pneumonia in the animals, resulting in the death of 53% of the animals. Triazavirin (sodium salt) showed a significant protective effect with a significant reduction in specific mortality of animals in the group experience (63%), as well as significant increases in life expectancy (3.1 days) compared with control animals receiving no drugs.

The studied analogs showed varying degrees of protective activity. One of them (BSS-14, the arginine salt of Triazavirin) was surprisingly more effective than Triazavirin (sodium salt). Some of the preparations (BSS4 and BSS11) showed approximately the same level of protection, while drugs BSS-64, BSS-65, mini289 and OIP-241 were less active than the reference drug, though, with the exception of UPI-241, had some level of protection from lethal influenza.

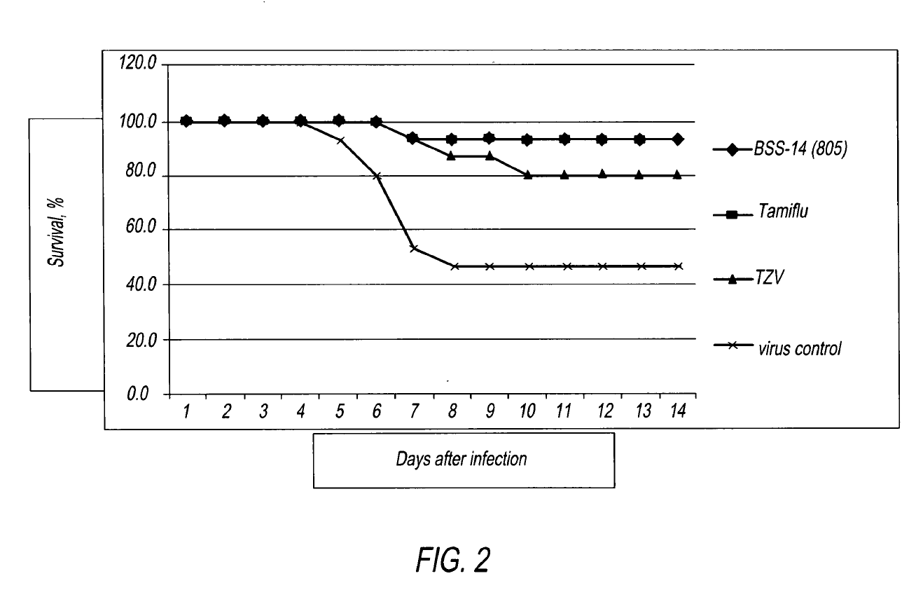

The increased effectiveness of BSS-14, the arginine salt of TZV, versus Tamiflu and the sodium salt of TZV is shown in FIG. 2.

Mice were inoculated with the virus at an LD50 dose and then treated with the various drugs shown. In the virus control group (no drug), as expected, 50% of the mice died within 8 days. TZV was able to rescue 80% of mice from infection while BSS14 (the arginine salt) gave a greater protective effect—equivalent to Tamiflu in this study.

Therefore the arginine salt form of TZV (BSS14) demonstrates greater efficacy compared to TZV in the absence of arginine.

TZV (sodium salt) and BSS14 (arginine salt of TZV), while comparable to Tamiflu in these studies, show better efficacy in vivo when delivered up to 3 days after infection with influenza virus. In comparison, Tamiflu has a narrower window in which it can be administered and must be administered within 24 hours of infection for greatest efficacy.

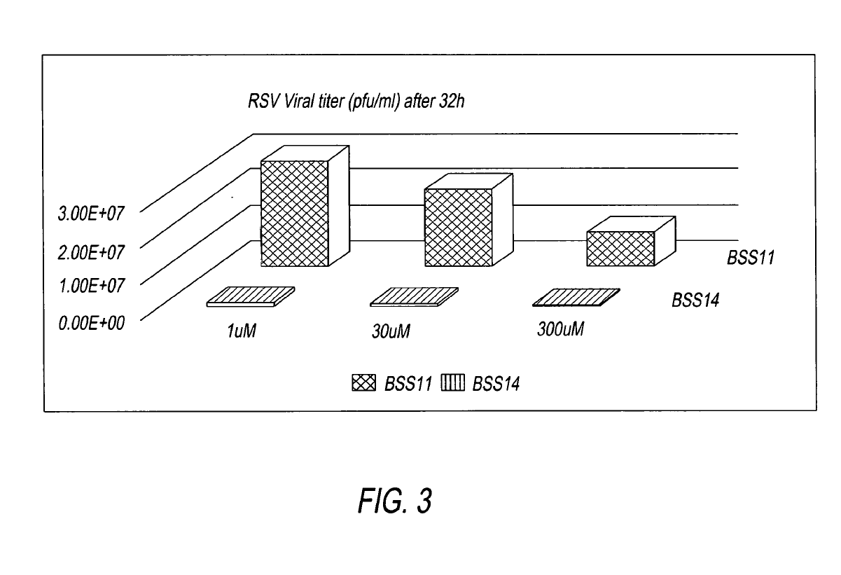

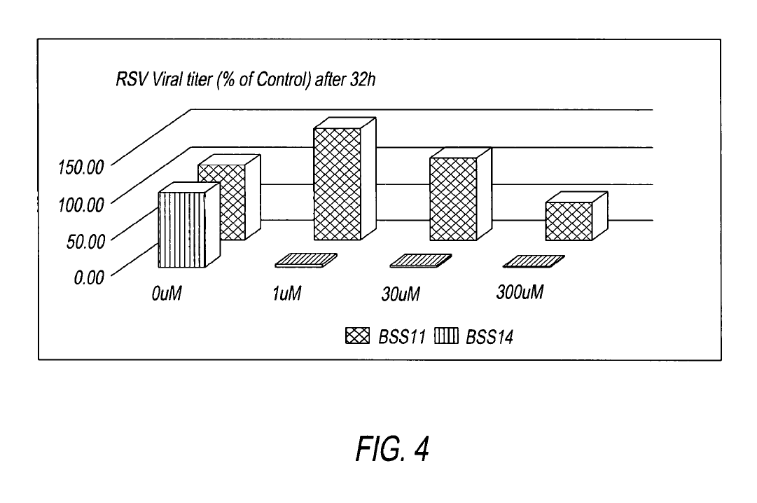

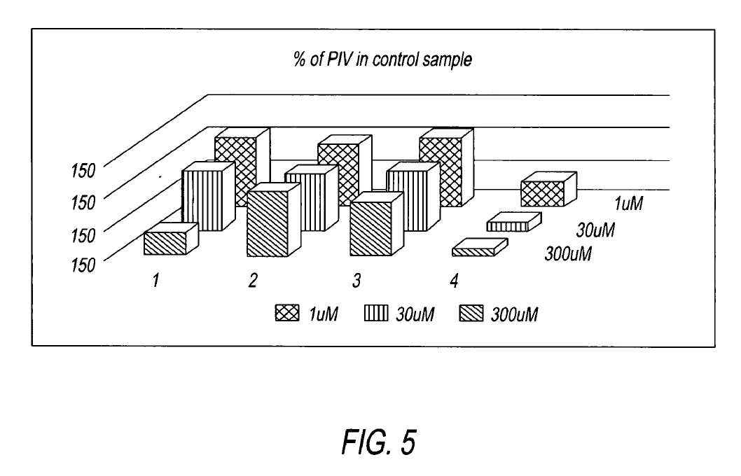

Additional data confirm the increased activity of the arginine salt of TZV (BSS14) against respiratory syncytial virus (RSV) and parainfluenza virus (PIV), as illustrated in FIG. 3-5. FIG. 3 shows a RSV Viral titer (pfu/ml) after 32h comparing BSS11 and BSS14. FIG. 4 shows RSV Viral titer (% of control) after 32 h comparing BSS11 and BSS14. FIG. 5 shows PIV Viral titer (% of control) after 32 h comparing TZV (1), BSS11 (2), 289 (3) and BSS14 (TZV arg salt; 4)

Novel Derivatives of TZV

The present invention also relates to novel derivatives of TZV identified by oxidation of the alkylthio derivatives into the corresponding sulfoxides 2a-d, 16-19 and sulfones 3a-d, as well as nucleophilic substitution of the nitro and sulfonyl groups by cysteine leading to compounds 5, 12-15, 20-21 as well as its analogs of the family of 2-alkylthio-6-nitro-1,2,4-triazolo-[5,1-c] [1,2,4] triazine-7-ones 1a-d, 8-11. Surprisingly, some of these derivatives show increased antiviral potency. These agents may be synthesized either as the final active form for treatment of viral infections or administered as the precursor that is then converted to these active forms. We claim the use of any means that will produce these moieties external or internal to the body as a means to treat viral infections. We further claim the methods used to manufacture these agents as detailed in the text below.

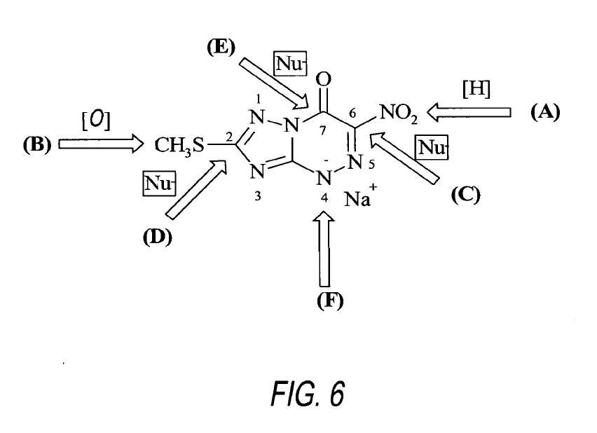

A number of derivatives of Formula 1 were examined (FIG. 6). One of them is redox transformations (A, B) either via reduction of the nitro group by reductases (A), or via oxidation of the alkyl fragment by oxydases followed by further transformations (B). Another opportunity is substitution of the alkyl and nitro-groups by transferases by action of N- and S-nucleophiles, such as lysine, arginine, and cysteine (C, D).

Hydrolytic ferments can cause transformations of TZV involving destruction of the triazine ring accompanied by the C—N bond cleavage. (E). Such a reaction was observed, for instance, on heating of 4-alkyl-6-nitro-1,2,4-triazolo[5,1-c]1,2,4-triazine-7-ones in water. Besides that, alkylation of the nitrogen atom is possible as well (F). Interaction of 6-nitro-1,2,4-triazolo[5,1-c]1,2,4-triazine-7-ones with alkyl halides or dimethyl sulfate affords the corresponding N-alkyl derivatives.

Oxidation of the alkyl groups by the singlet oxygen and its other derivatives (O⁻, NO⁻, H₂O₂, ONOO⁻, HO⁻) were also considered, particular with relationship to the formation of nitro-peroxide radicals ONOO⁻, which are actively generated during viral infections. The influenza virus and other pathogenic agents are able to induce INOS (induced NO-synthetase) synthesis, thus resulting in the formation of an excess of NO in both tissues and peripheral blood. Under active production of oxygen radicals, most of NO is transformed into the nitroperoxide anion. The oxidation of the S-methyl group in TZV was considered as well as activation of the nitro group in the 6-position of TZV followed by its transformation into unstable nitrosyl radical.

In another embodiment, the present invention relates to oxidation of the S-methyl group and behaviour of oxidation products by action of S- and N-nucleophiles.

Redox Transformation of TZV and its Derivatives

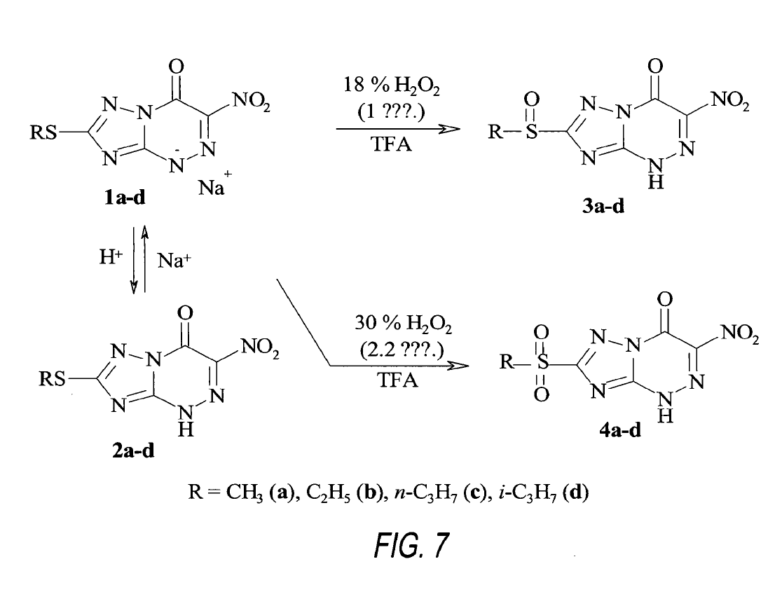

The starting point of this research was the synthesis of model compounds by means of oxidation of the alkylthio group in sodium salts of 2-alkylthio-6-nitro-1,2,4-triazolo [5,1-c] [1,2,4]-triazine-7-ones 1a-d and related N—H derivatives 2a-d, thus leading to the formation of heterocyclic alkylsulfoxides 3a-d and alkylsulfones 4a-d. Sulfoxides 3a-d were obtained by treatment of compounds 1a-d or 2a-d with an equimolar amount of 18% hydrogen peroxide in trifluoroacetic acid (FIG. 7).

The structure of compounds 3a-d was determined by NMR and IR spectroscopy as well as elemental analysis. The ¹H NMR spectra of alkylsulfoxides 3a-d show characteristic signals of the alkyl groups R, as well as a broadened one-proton signal of NH at δ=7.46+11.15 ppm.

Being compared to the ¹H NMR spectra of the starting compounds 1a-d, sulfoxides 3a-d show downfield shifts for protons of the S—CH fragments (Δδ=0.3+0.4 ppm), The ¹³C NMR spectra of triazolo-1,2,4-triazines 3a-d exhibit the signals of alkyl groups in the region δ=6-54 ppm, and the resonance peaks of heterocyclic carbons. The IR spectra of compounds 3a-d show the characteristic bands corresponding to stretching vibrations of the sulfoxide group (v S═O 992+1036 sm⁻¹).

Further oxidation of 3a-d into the corresponding alkylsulfones 4a-d was accomplished in 62-71% yields by gradual addition of 2.2 equivalents of 30% H₂O₂ to a suspension of 2-alkylthio -1,2,4-triazolo [5,1-c] triazines 1a-d in trifluoroacetic acid at room temperature (FIG. 2).

The ¹H NMR spectra of compounds 4a-d exhibit pronounced downfield shifts for the S-alkyl resonance signals, compared to those of the starting materials 1. The IR spectra of 4a-d contain the characteristic absorption bands, corresponding to stretching vibrations of the sulfonyl group (vₐₛ SO₂ 1293+1347 sm⁻¹ and vₛ SO₂ 1135+1140 sm⁻¹).

### Nucleophilic Substitution of the Methylsulfonyl Group

Our data demonstrate that the NO₂ group attached to an aromatic (heteroaromatic) ring is prone to undergo displacement by action of nucleophiles. Indeed, the structure of 2-alkylsulfonyl-6-nitro-1,2,4-triazolo-[5,1-c]-[1,2,4]triazine-7-ones 4a-d contains two groups that are prone to nucleophilic displacement reactions. This is important for studying of comparative reactivity of two good leaving groups: CH₃SO₂— in the 1,2,4-triazole ring and NO₂— in the 1,2,4-triazine ring of compounds 3a-d, as well as for modelling of metabolic behaviour of TZV. In this study, we have employed S-containing compounds (cysteine and cysteamine), as nucleophilic reagents. These compounds can be considered as models of protein fragments, containing the cysteine moiety, and, on the other hand, as biogenic fragments, which might be incorporated in the structure of 1,2,4-triazolo [5,1-c]-1,2,4-triazines.

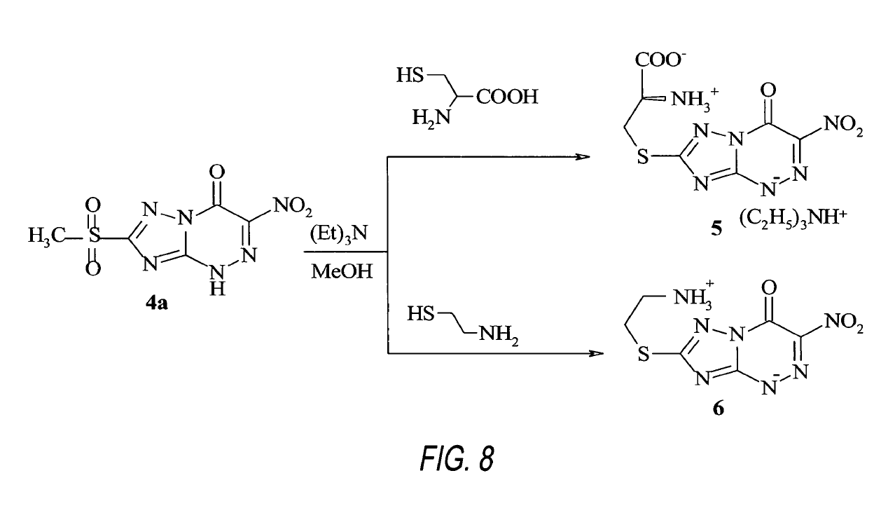

Substitution of the sulfonyl group in 2-methylsulfonyl-1,2.4-triazolo [5,1-c]-1,2,4-triazine 4a by action of both cysteine and cysteamine takes place on reflux in dry methanol and requires several days in the presence of triethylamine to give substitution products 5 and 6 in 41-46% yields (FIG. 8).

The data of NMR, IR and elemental analysis for compounds 5 and 6 provide evidence for their structure. The H¹ NMR spectrum of triethylammonium salt of 2′-amino-2′-5 carboxy-ethylthio triazolotriazine 5 exhibit signals of the cysteine moiety with characteristic splitting for the signals of SCH₂ protons, each of which is represented as a double doublet (δ=3.65 ppm., ⁿJ=15.2 and 7.5; δ=3.98 ppm., ⁿJ=15.2 and 3.8); as well as the signals of three ethyl groups of the triethylammonium cation. The ¹H NMR spectrum of compound 6 shows characteristic triplets of the cysteamine moiety due to zwitter ion formation. Substitution of the methylsulfonyl fragment is evidenced by the absence of its signal in the ¹H NMR spectra at 3.27 ppm by disappearance of characteristic absorption bands of the sulfonyl group in the IR spectra, as well as by the presence of characteristic frequencies of the nitro group (v NO₂ 1504+1505 cm⁻¹ and 1361+1375 cm⁻¹).

### Nucleophilic Substitution of the Nitro Group

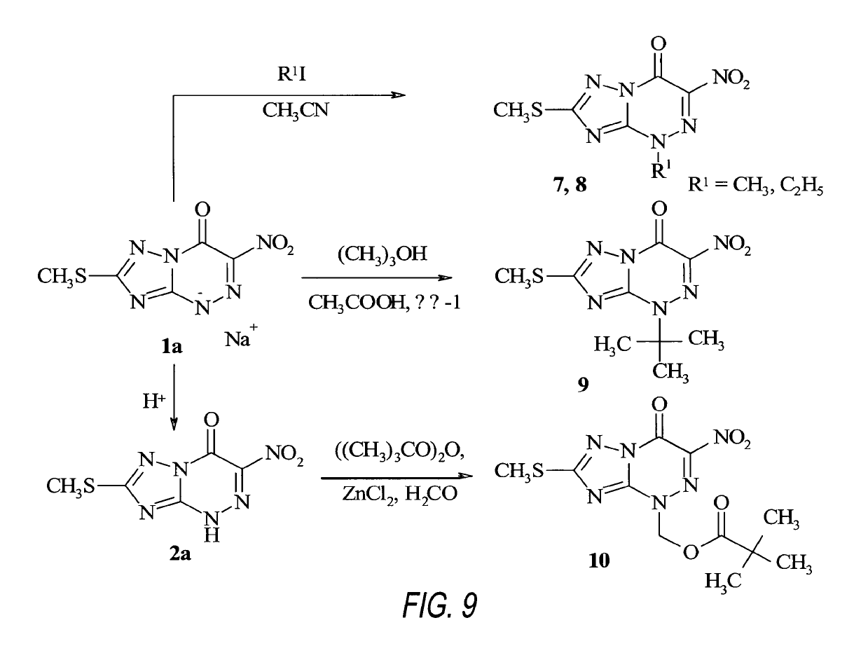

We have synthesized 4-alkyl-2-methylthio-6-nitro-1,2,4-triazolo[5,1-c]1,2,4-triazines 7-10 (FIG. 9) and their related sulfones 11-14 to elucidate competitive substitutions of sulfonyl and nitro groups. 4-Methyl and 4-ethyl-6-nitro-1,2,4-triazolo[5,1-c][1,2,4]triazine-7-ones, 7 and 8, were prepared by using the procedure developed earlier in our laboratory. 2-Methylthio-4-tert-butyl-6-nitro-1,2,4-triazolo[5,1-c]1,2,4-triazine-7 9 was synthesized by treatment of the sodium salt 1a with tent-butyl alcohol in trifluoroacetic acid in the presence of resin KU-1. In order to obtain compound 10, a new effective method for incorporation of the pivaloyloxymethyl moiety in the position 4 of triazolo[5,1-c] [1,2,4] triazine has been developed. It involves conversion of the sodium salt 1a into NH-acid 2a, followed by the reaction in melt with pivaloyl anhydride, paraformaldehyde and zinc chloride at 140° C. (FIG. 9).

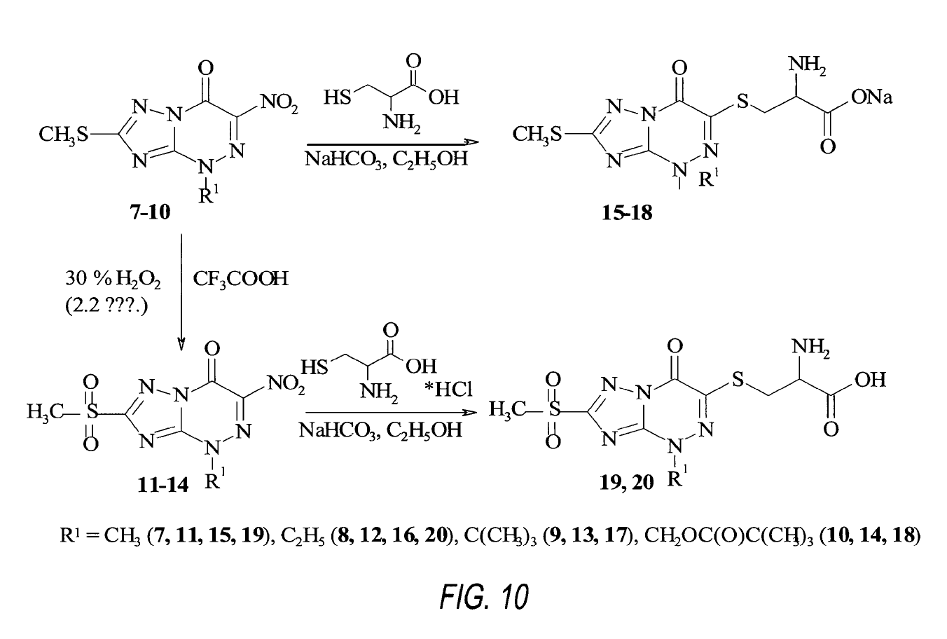

We show that 2-methylthio-4-R′-6-nitro-1,2,4-triazolo[5,1-c]-1,2,4-triazine-7-ones 7-10, das well as compounds 1a-d, are readily oxidized into the corresponding sulfones 11-14 with an excess of hydrogen peroxide (FIG. 10). The ¹H NMR spectra of compounds 11-14 are in full correspondence with their structures, showing the signals of the methylsulfone groups (δ=3.0+3.5 ppm), as well as the presence of N-alkyl groups. The IR spectra contain the characteristic absorption bands of carbonyl, nitro, and sulfonyl groups.

In order to determine an opportunity of interaction of TZV with N- and S-containing fragments of proteins, we have exploited cysteine as a reference nucleophile. Interaction of compounds 7-10 -6-(2′-amino-2′-carboxyethyl)-1,2,4-triazolo[5,1-c]-1,2,4-triazine-7-ones 15-18 in 27-68% yields (FIG. 10). Since the methylthio group in compounds 7-10 is a more inert one that the nitro group, it does not enter the substitution reaction under these conditions.

It should be noted that, in spite of two plausible competitive routes for the reaction of triazolo -1,2,4-triazines 11 and 12 with cysteine, only substitution of the nitro group takes place at room temperature to give compounds 19 and 20, without displacement of the methylsulfonyl group (FIG. 10), as evidenced by the data of ¹H NMR and IR spectroscopy.

Preparation of 1,2,4-triazolo[5,1-c]-1,2,4-triazines with the cysteine moiety indicates at an opportunity for enzymatically catalysed synthesis of such compounds. Such transformations are in agreement with the surprising ability of the compounds to undergo S-guanylation reactions similar to what occurs in components of the signal redox system and modification of cellular and viral proteins by 8-nitroguanosine.

Antiviral Activity of S-oxidation Products and Nucleophilic Substitution of the Nitrogroup

To establish whether the S-oxidation products of TZV and their derivatives 1a-d might have antiviral activity, a number of biological tests in vitro have been performed for all of the compounds including 1a, 3a-d, 4a-d and 5-18 (Table 2).

As it can be seen from Table 2, the majority of compounds studied exhibit antiviral activity. However, S-oxidation of compounds la-d did not produce any significant enhancement of antiviral activity in these in vitro experiments.

Biological trials of cysteine derivatives (compounds 12-15) have shown that these compounds surprisingly exhibit a high antiviral activity in vitro compared to nitro derivatives of azolo-1,2,4-triazines.

TABLE 2
Biological tests of compounds 1a, 2a-d, 3a-d, 5 and 10-19

| | | | Antiviral activity | |
|---|---|---|---|---|
| | Minimal | Working | A(H1N1)* | A(H3N2)* |
| | toxic dose | concentration, | A/Puerto | A/Victoria/ |
| Compound | □g/ml | □g/ml | Rico/8/34 | 35/72 |
| 1a | >400 | 100 | 2.0 | 2.25 |
| 3a | >200 | 100 | 1.0 | 1.0 |
| 3b | >200 | 100 | 0.5 | 0.5 |
| 3c | >200 | 100 | 1.0 | 1.0 |
| 3d | >200 | 100 | 1.0 | 1.0 |
| 4a | >200 | 100 | 0.0 | 0.0 |
| 4b | >200 | 100 | 0.5 | 0.5 |
| 4c | >200 | 100 | 0.0 | 0.0 |
| 4d | >200 | 100 | 1.0 | 1.0 |
| 5 | 200 | 100 | 0.0 | 0.5 |
| 6 | 200 | 100 | 0.0 | 0.5 |
| 7 | >100 | 100 | 1.0 | 0.0 |
| 8 | >200 | 100 | 1.0 | 0.0 |
| 9 | 100 | 50 | 2.0 | 1.5 |
| 10 | 100 | 50 | 1.0 | 1.0 |
| 11 | >100 | 100 | — | 0.0 |
| 12 | >100 | 100 | — | 0.5 |
| 13 | >100 | 100 | — | 0.5 |
| 14 | >100 | 100 | — | 1.0 |
| 15 | >100 | 100 | — | 0.0 |
| 16 | >100 | 100 | — | 0.0 |
| 17 | 200 | 125 | 0.0 | 0.0 |
| 18 | 200 | 62.5 | 3.0 | 3.5 |

*reduction in infectivity of the virus, Ig ID_{50/20 □l}

Incorporation of the cysteine moiety in TZV structure (compound 18) surprisingly results in a significant enhancement of its antiviral activity.

### Experimental Details

NMR—The NMR spectra were recorded on a Bruker DRX-400 instrument in DMSO-d₆, CDCl₃, D₂O. ¹H NMR spectra were recorded at 400 MHz with TMS as an internal standard and ¹³C NMR spectra at 100 MHz, respectively. The elemental analysis (C, H, N) was carried out on a Perkin Elmer 2400-II CHNSIO. IR spectra were obtained using a Perkin Elmer Spectrum One B FTIR in a thin layer (DRA). The course of the reactions was monitored, and the purity of the products was checked by TLC on Sorbfil (Russia) in ethyl acetate and butanol-acetic acid-water 4:1:1.

Antiviral Assays—To assess antiviral activity, the microtetrazolium (MTT) test was chosen, which allows one to characterize antiviral activity by the viral cytopathic effect. Virus replication in the presence of the compounds under test was studied by estimating the cytopathic effect in the cell culture. Cell viability was estimated using MTT, which is used to characterize the intensity of mitochondrial respiration of live cells. To perform this, 100 μl of 3-(4,5-dimethyl -thiazolyl-2)-tetrazolium bromide (5 mg/ml) (ICN Biochemicals Inc., Aurora, Ohio) was added to the wells. The optical density in the plates' wells was measured using a Hydex Chameleon reader. The antiviral effect was expressed as reduction in the infectious viral titre (ID₅₀) compared to the virus control (the well with appropriate virus dilution) without preparations). ID₅₀ is calculated based on the linear regression equations of the control and test wells over all viral titres in the MTT test. ID₅₀=(y50%-a)/b, y50%—optical density of cell control/2; a—intersection of regression lines and y-axis; b—line regression coefficient). The inhibition degree (%) is the calculated estimation criterion.

The synthesis of sodium salts of 2-alkylthio-6-nitro-1,2,4-azolo[5,1-c]1,2,4-triazine-7-ones dihydrates (1a-d) has been reported previously.

2-iso-Propylthio-6-nitro-1,2,4-triazolo[5,1-c] 1,2,4-triazine-7-one dihydrate (1d) was obtained from 3-iso-propylthio-5-amino-1,2,4-triazole using the previously reported procedure. Yellow crystal powder, mp 185° C., yield 49%, mp 185° C., ¹H NMR (DMSO-d₆, 400 MHz): 3.89 (1H, m, CH), 1.40 (6H, d, J=6.52, 2CH₃); ¹³C NMR (DMSO-d₈, 100 MHz): 165.43 (C₂), 160.42 (C₃ₐ), 145.03 (C₆), 143.54 (C₇), 36.72 (CH), 23.74 (2CH₃): IR (v/sm⁻¹): 1671 (C═O); 1510, 1363 (NO₂), Element. anal. calc. for C₇H₇N₈NaO₃S*2H₂O, %: C—26.75, H—3.53, N—26.74, found, %: C—26.81, H—3.66, N—26.58.

Procedure 1 for the synthesis of compounds 2-alkylsulfinyl-6-nitro-1,2,4-triazolo[5,1-c]1,2,4-triazine-7-ones (3a-d). Hydrogen peroxide 18% (1.78 ml) was added to a stirred suspension of 2-alkylthio-6-nitro-1,2,4-triazolo[5,1-c]1,2,4-triazine-7-one sodium salt (1) (0.01 mol) in trifluoroacetic acid (14 ml). After 3 h of stirring at room temperature, the residue was filtered and recrystallized from iso-propanol.

2-Methylsulfinyl-6-nitro-1,2,4-triazolo[5,1-c]1,2,4-triazine-7-one (3a) was obtained from 1a using Procedure 1. Beige crystal powder, yield 70%, mp 256° C., ¹H NMR (DMSO-d₆, 400 MHz): 7.46 (1H, br s, NH), 3.06 (3H, s, CH₃); ¹³NMR (DMSO-d₅, 100 MHz): 163.86 (C₂), 158.41 (C₃ₐ), 144.69 (C₆), 143.64 (C₇), 41.84 (CH₃SO); IR (v/sm⁻¹): 1750 (C═O); 1036 (—SO—); 1553, 1340 (NO₂); Element. anal. calc. for C₅H₄N₈O₄S, %: C—24.59, H—1.65, N—34.42, found, %: C—24.62, H—1.43, N—34.28.

2-Ethylsulfinyl-6-nitro-1,2,4-triazolo[5,1-c]1,2,4-triazine-7-one (3b) was obtained from 1b using Procedure 1. Beige crystal powder, yield 74%, mp 227° C., ¹H NMR (DMSO-d₆, 400 MHz): 11.15 (1H, br s, NH), 3.26 (2H, m, CH₂), 1.23 (3H, t, J=7.4, CH₃); ¹³0 NMR (DMSO-d₆, 100 MHz): 168.34 (C₂), 157.58 (C₃ₐ), 144.37 (Cₛ), 143.87 (C₇), 46.65 (CH₂SO), 6.16 (CH₃); IR (v/sm⁻¹): 1748 (C═O); 1022 (—SO—); 1552, 1336 (NO₂); Element. anal. calc. for C₈H₈N₆O₄S, %: C —27.91, H—2.34, N—32.55, found, %: C—27.87, H—2,27, N—32.31.

2-Propylsulfinyl-6-nitro-1,2,4-triazolo[5,1-c]1,2,4-triazine-7-one (3c) was obtained from 1c using Procedure 1. Beige crystal powder, yield 72%, mp 222° C., ¹H NMR (DMSO-d₆, 400 MHz): 8.41 (1H, br s, NH), 3.23 (2H, m, SOCH₂), 1.56-1.76 (2H, m, CH₂), 1.01 (3H, m, CH₃); ¹³C NMR (DMSO-d₆, 100 MHz): 168.19 (C₂), 157.87 (C₃ₐ), 144.19 (C₈), 143.46 (C₇), 54.15 (CH₂SO), 15.22 (CH₂), 12.95 (CH₃); IR (v/sm⁻¹): 1748 (C═O); 1013 (—SO—); 1556, 1336 (NO₂); Element. anal. calc. for C₇H₈N₈O₄S, %: C—30.88, H—2.96. N—30.87, found, %: C—30.82. H—3.12, N 30.77.

2-iso-Propylsulfinyl-6-nitro-l2,4-triazolo[5,1-c]1,2,4-triazine-7-one (3d) was obtained from 1d using Procedure 1. Beige crystal powder, yield 77%, mp 239° C., ¹H NMR (DMSO-d₆, 400 MHz): 7.59 (1H, br s, NH), 3.43 (1H, m, CH), 1.27 (6H, dd, J=6.8, 2CH₃); ¹³C NMR (DMSO-d₆, 100 MHz): 167.78 (C₂), 157.43 (C₃ₐ), 144.33 (C₈), 143.86 (C₇), 53.08 (CHSO), 15.93 (CH₃), 14.60 (CH₃); IR (v/sm⁻³); 1752 (C═O), 992(—SO—); 1555, 1342 (NO₂); Element. anal. calc. for C₇H₈N₈O₄S, %: C—30.88, H—2.96, N—30.87, found, %: C—31.07, H—2.95, N—30.89.

Procedure 2 for the synthesis of 2-alkylsulfonyl-6-nitro-1,2,4-triazolo[5,1-c]'1,2,4-triazine -7-ones (4a-d). Hydrogen peroxide 30% (4 ml) was added dropwise to a suspension of 2-alkylthio-6-nitro-1,2,4-triazolo[5,1-c]1,2,4-triazine-7-one sodium salt under stirring 1) (0.01 mol) in trifluoroacetic acid (14 ml), with the temperature not exceeding 80° C. After 3 h of stirring at room temperature, the resulting precipitate was filtered off and recrystallized from iso-propanol.

2-Methylsulfonyl-6-nitro-1,2,4-triazolo[5,1-c]1,2,4-triazine-7-one (4a) was obtained from 1a using Procedure 2. Beige crystal powder, yield 66/%, mp 275° C., ¹H NMR (DMSO-d₈, 400 MHz): 12.28 (1H, br s, NH), 3.27 (3H, s, CH₃); ¹³C NMR (DMSO-d₆, 100 MHz): 169.06 (C₂), 157.36 (C₃ₐ), 144.07 (Cₛ), 143.57 (C₇), 39.28 (01-1₃50₂): IR (v/sm⁻¹): 1759 (0=0); 1347, 1138 (—SO₂); 1570, 1323 (NO₂): Element. anal. calc. for C₅H₄N₈O₅S, %: C—23.08, H—1.55, N—32.30, found, %: C—23.21, H—1.31, N—32.33.

2-Ethylsulfonyl-6-nitro-1,2,4-triazolo[5,1-c]1,2,4-triazine-7-one (4b) was obtained from 1b using Procedure 2. Beige crystal powder, yield 64%, mp 259° C., ¹H NMR (DMSO-d₆, 400 MHz): 9.71 (1H, br s, NH), 3.51 (2H, q, J=7.4, CH₂), 1.31 (3H, t, J=7.4, CH₃); ¹³C NMR (DMSO-d₆, 100 MHz): 162.82 (C₂), 158.93 (C₃ₐ), 145.06 (C₆), 144.02 (C₇), 48,54 (CH₂SO₂), 7,26 (CH₃); IR (v/sm⁻¹): 1759 (C═O); 1311, 1140 (—SO₂—); 1557, 1332 (NO₂); Element. anal. calc. for C₆H₈N₈O₅S, %: C—26.28, H—2.21, N—30.65, found, %; c—26.44, H—2.20, N—30.43.

2-Propylsulfonyl-6-nitro-1,2,4-triazolo[5,1-c]1,2,4-triazine-7-one (4c) was obtained from 1c using Procedure 2. Beige crystal powder, yield 71%, mp 264° C., ¹H NMR (DMSO-d₆, 400 MHz): 11.98 (1H, br s, NH), 3.52 (2H, t, J=7.6, SO₂CH₂), 1.71 (2H, m, CH₂), 0.98 (3H, t, J=7.4, CH₃); ¹³C NMR (DMSO-d₆, 100 MHz): 163.22 (C₂), 158.45 (C₃ₐ), 144,91 (C₈), 143.99 (C₇), 55.35 (CH₂SO₂), 16.20 (CH₂), 12.95 (CH₃); IR (v/sm⁻¹): 1748 (C═O); 1293, 1139 (—SO₂—); 1556, 1326 (NO₂); Element. anal. calc. for C₇H₈N₈O₅S, %: C—29.17, H—2.80, N—29.16, found, %, C—29.01, H—2.88, N—29.24.

2-iso-Propylsulfonyl-6-nitro-1,2,4-triazolo[5,1-c]1,2,4-triazine-7-one (4d) was obtained from 1d using Procedure 2. Beige crystal powder, yield 62%, mp 282 ^{G}C, ¹H NMR (DMSO-d₆, 400 MHz): 8.26 (1H, br s, NH), 3.67 (1H, m, CH), 1.34 (6H, d, J=6.8, 2CH₃); ¹³C NMR (DMSO-d₆, 100 MHz): 161.91 (0₂), 158.80 (C₃ₐ), 144.98 (C₆), 144.03 (C7), 54.30 (CHSO₂), 15.03 (20H₃); IR (v/sm⁻¹): 1749 (C═O): 1311, 1135 (—SO₂—); 1556, 1326 (NO₂); Element. anal. calc. for C₇H₈N₈O₅S, %: C—29.17, H—2.80, N—29.16, found, %: C—29.11, H—2.69, N—29.00.

Procedure 3 for the nucleophilic substitution in 2-methylsulfonyl-6-nitro-1,2,4-triazolo[51-c]1,2,4-triazine-7-one. Triethylamine was added to a suspension of 0.001 mol cysteine (or cysteinamine) in 20 ml of methanol. After 5 min of stirring in argon atmosphere, equivalent of 2-alkylsulfonyltriazolotriazine was added and the reaction mixture was refluxed. The completion of the reaction was confirmed by TLC in butanol-acetic acid-water system 4:1;1, the reaction mixture was evaporated, and the residue was washed with iso-propanol and purified.

Triethylammonium salt of (2′-amino-2′-carboxyethylthio)-6-nitro-1,2,4-triazolo[5,1-c][1,2,4]triazine hydrate 5 was obtained using Procedure 3 and 3 eq of triethylamine. The product was recrystallized from ethanol (aq). Bright yellow crystal powder, yield 46%, mp 158 ° C. ¹H NMR (D₂O DMSO-d₆, 400 MHz): 4.27 (1H, dd, J=7.5, 3.8, CHN), 3.98 (1H, dd, J=15.2, 3.8, Hₐ in SCH₂), 3.65 (1H, dd, J=15.2, 7.5, H_{b} in SCH₂), 3.21 (6H, q, J=7.28, 3CH₂), 1.29 (9H, t, J=7.28, 3CH₃); ¹³C NMR (D₂O, 100 MHz): 172.02 (COD), 166.09 (C₂), 159.16 (C₃ₐ), 144.55 (C₈), 143.06 (C₇), 54.48 (CHN), 46.72 (3CH₂), 31.68 (SCH₂), 8.27 (3CH₃); IR (v/sm⁻¹): 1682, 1615 (C═O); 1504, 1361 (NO₂); Element. anal. calc. for C₁₃H₂₂N₈O₅S*H₂O, %: C—37.10, H—5.71, N—26.63, found, %: C—36.96, H—5.56, N—26.72.

2-(2′-Amino-ethylthio)-6-nitro-1,2,4-triazolo[5,1-c][1,2,4]triazine hydrate 6 was obtained using Procedure 3 and 2 eq of triethylamine. The product was recrystallized from methanol (aq), Bright yellow crystal powder, yield 41%, mp 285° C., ¹H NMR (D₂O, 400 MHz): 3.57 (2H, t, J=7.03, NCH₂), 3.49 (2H, t, J=7.03, SCH₂); ¹³C NMR (DMSO-d₅, 100 MHz): 163.95 (C₂), 160.13 (C₃ₐ), 144.77 (C₆), 142.94 (₇), 38.70 (CH₂N), 27.88 (SCH₂); IR (v/sm⁻¹): 1690 (C═O); 1514, 1371 (NO₂); 3521 (broadened) (—NH₃⁺); Element. anal. calc. for C₈H₇N₇O₃S*H₂O, %: C—26.18, H—3.27, N—35.64, found, %: C —25.91, H—2.98, N—35.57.

The synthesis of 2-methylthio-4-alkyl-6-nitro-1,2,4-triazolo[5,1-c]1,2,4-triazine-7-ones (7,8) has been reported previously.

The synthesis of 2-methylthio-4-tert-butyl-6-nitro-1,2,4-triazolo[5,1-c]1,2,4-triazine-7-one 9. Cation-exchange resin KU-1 and tert-butanol (eq) were added to a suspension of triazolotriazine (0.01 mol) 1a in trifluoroacetic acid (15 ml). The reaction mixture was stirred at room temperature for several days until complete dissolution of 1a. After completion, water (30-40 ml) was added and the suspension was filtered. The residue was washed with water. The product is yellow crystal powder of 9, yield 96%, mp 157° C., ¹H NMR (CDCl₃, 400 MHz): 2.73 (3H, s, SCH₃), 1.83 (9H, s, 3CH₃); ¹³C NMR (DMSO-d₆, 100 MHz): 166.52 (C₂), 151.60 (C₃ₐ), 142.36 (C₆), 139.75 (C), 69.97 (C(CH₃)₃), 27.45 (3CH₃), 14.10 (SCH₃); IR (v/sm⁻¹): 1333, 1542 (NO₂), 1731 (C═O); Element. anal. calc. for C₉H₁₂N₈O₃S, %: C—38.02, H—4.25, N—29.56, found, %: C —38.02, H—4.52, N—29.69.

The synthesis of 2-methylthio-4-pivaloyloxymethyl-6-nitro-1,2,4-triazolo[5,1-c]1,2,4-triazine-7-one (10). Triazolotriazine NH-acid 1a (1.13 g), pivalic acid anhydride (2 ml, 2 eq), paraform (0.3 g, 2 eq) and ZnCl₂ in catalytic quantity were heated at 140° C. for 3 h. The reaction mixture was cooled at room temperature. The product was extracted with CHCl₃ (20 ml), and the organic layer was washed with water (2×10 ml) and dried over Na₂SO₄. The solvent was evaporated, and the residue was left overnight in hexane and then filtered. The product is yellow crystal powder of 10, yield 82%, mp 80° C. ¹H NMR (CDCl₃, 400 MHz): 6.25 (2H, s, CH₂N), 2.72 (3H, s, SCH₃), 1.23(9H, s, 3CH₃); ¹³C NMR (DMSO-d₆, 100 MHz): 176.28 (C═O), 167.43 (C₂), 151.93 (C₃ₐ), 142.40 (C₆), 142.14 (C₇), 74.88 (CH₂N), 38.87 (C(CH₃)₃), 26.98 (3CH₃), 14.10 (SCH₃); IR (v/sm⁻¹): 1359, 1573 (NO₂), 1744 (C═O); Element, anal. calc. for C₁₁H₁₄N₈O₆S, %: C —38.59, H—4.12, N—24.55, found, %: C—38.69, H—3.99, N—24.42.

Procedure 4 for the synthesis of 2-methylsulfonyl-4-alkyl-6-nitro-1,2,4-triazolo[5,1-c]1,2,3-triazine-7-ones (11-14). To a stirred suspension of 2-methylthio-4-alkyl-6-nitro-1,2,4-triazolo[5,1-c]1,2,3-triazine-7-one (0.002 mol) (7-10) in trifluoroacetic acid (3 ml) hydrogen peroxide (1.6 ml, 30%) was added dropwise. The reaction mixture was stirred at room temperature for 3 h, the residue was filtered and recrystallized from ethyl acetate.

2-Methylsulfonyl-4-methyl-6-nitro-1,2,4-triazolo[5,1-cy]1,2,3-triazine-7-one (11) was obtained from 7 using Procedure 4. The product is beige crystal powder, yield 63%, mp 242° C., ¹H NMR (DMSO-d₆, 400 MHz): 4.18 (3H, s, NCH₃), 3.60 (3H, s, SO₂CH₃); ¹³ C NMR (DMSO-d₆, 100 MHz): 163.76 (C₂), 153,08 (C₃ₐ), 143.32 (C₆), 141.56 (C₇), 43.27 (NCH₃), 42.14 (SO₂CH₃); IR (v/m⁻¹): 1745 (C═O): 1314, 1153 (—SO₂); 1560, 1330 (NO₂); Element. anal. calc. for C₈H₈N₈O₅S, %: C—26,28, H—2.1, N—30.65, found, %: C—26.42, H—2.35, N—30,62.

2-Methylsulfonyl-4-ethyl-6-nitro-1,2,4-triazolo[5,1-c]1,2,3, -triazine-7-one (12) was obtained from 8 using Procedure 4. The product is beige crystal powder, yield 57%, mp 190° C., ¹H NMR (DMSO-d₆, 400 MHz): 4.53 (2H, q, J=7.28, NCH₂), 3.51 (3H, s, SO₂CH₃), 1.55 (3H, t, J=7.28, CH₃); ¹³C NMR (DMSO-d₆, 100 MHz): 163.71 (C₂), 152.61 (C₃ₐ), 143.34 (C₆), 141.75 (C₇), 51.63 (CH₂N), 42.17 (SO₂CH₃), 13.19 (CH₃); IR (v/sm⁻¹): 1747 (C═O); 1316, 1155 (—SO₂—); 1567, 1329 (NO₂); Element. anal. calc. for C₇H₆O₅S, %: C—29.17, H—2.80, N—29.16, found, %: C —29.31, H—2.82, N—29.14.

2-Methyisulfonyl-4-tert-butyl-6-nitro-1,2,4-triazolo[5,1-c]1,2,3-triazine-7-one (13) was obtained from 9 using Procedure 4. The product is beige crystal powder, yield 82%, mp 275° C., ¹H NMR (DMSO-d₆, 400 MHz): 3.51 (3H, s, SO₂CH₃), 1.80 (9H, s, 3CH₃); ¹³C NMR (DMSO-d₆, 100 MHz): 163.12 (C₂), 151.81 (C₃ₐ), 143.25 (C₈), 139.90 (C₇), 70.95 (C(CH₃)₃), 42.12 (SO₂CH₃), 27.51 (3CH₃); IR (v/sm⁻¹): 1746 (C═O); 1317, 1149 (—SO₂—); 1556, 1331 (NO₂); Element. anal. calc. for C₉H₁₂N₆O₅S, %: C—34.18, H—3,82, N—26.57, found, %: C—34,41, H —3.63, N—26.41.

2-Methylsulfonyl-4-pivaloyloxymethyl-6-nitro-1,2,4-triazolo[5,1-c]1,2,3-triazine-7-one (14) was obtained from 10 using Procedure 4. The product is beige crystal powder, yield 79%, mp 186° C., ¹H NMR (DMSO-d₆, 400 MHz): 6.29 (2H, s, CH₂N), 3.50 (3H, s, SO₂CH₃), 1.20 (9H, s, 3CH₃); ¹³C NMR (DMSO-d₆, 100 MHz): 176.27 (C═O), 163.67 (C₂), 152.16 (C₃,), 143.06 (Cₑ), 142.42 (C₇), 74.98 (CH₂N), 42.11 (SO₂CH₃), 38.87 (C(CH₃)₃), 26.94 (3CH₃); IR (v/sm⁻¹): 1751 (C═O); 1320, 1154 (—SO₂—); 1566, 1320 (NO₂); Element. anal. calc. for C₁₁H₁₄N₆O₆S, C —35.30, H—3.77, N—22.45, found, 9/0: C—35,36, H—3.59, N—22.61.

Procedure 5 for the synthesis of 2-methylthio-4-alkyl-6-(2′-amino-2′-carboxyethylthio) -1,2,4-triazolo[5,1-c]1,2,4-triazine-7-ones (15-18). Triazolotriazine (7-10) was dissolved in absolute ethanol and added to a stirred fresh solution of cysteine (1 eq) and sodium bicarbonate (2 eq, 1 M). The reaction mixture was stirred for 1 h at room temperature, the residue was filtered and recrystallized from ethanol (aq).

Sodium salt of 2-methylthio-4-methyl-6-(2′-amino-2′-carboxyethylthio)-1,2,4-triazolo[5,1-c]1,2,4-triazine-7-one hydrate (15) was obtained from 7 using Procedure 5. The product is beige crystal powder, yield 62%/, mp 185° C., ¹H NMR (D₂O, 400 MHz): 4.18 (1H, dd, J=8.0, 4.2, CHN), 4.07 (3H, s. CH₃), 3.88 (1H, dd, J=15.0, 4,2, Hₐ in SCH₂), 3,57 (1H, dd, J=15.0, 8.0, H_{b} in SCH₂), 2.68 (3H, s, SCH₃); ¹³C NMR (D₂O+CF₃oCOOD, 100 MHz): 169.67 (COONa), 168.65 (C₂), 151.40 (C₃ₐ), 146.93 (C₇), 141.32 (C₈), 51.54 (CHN), 41.48 (CH₃N), 29.25 (SCH₂), 13.20 (SCH₃); IR (v/sm⁻¹): 1698 (C═O); Element. anal. calc. for C₉H₁₂N₈NaO₃S₂*H₂O, %: —30.33, H—3.68, N—23.58, found, %: C—30.58, H—3.99, N—23.75.

Sodium salt of 2-methylthio-4-ethyl-6-(2amino-2′-carboxyethylthio)-1,2,4-triazolo[5,1-c]1,2,4-triazine-7-one hydrate (16) was obtained from 8 using Procedure 5. The product is white crystal powder, yield 68%, mp 210° C., ¹H NMR (D₂O, 400 MHz): 4.45 (2H, q, J=7.1, CH₂), 4.17 (1H, dd, J=8.4, 4.1, CHN), 3.93 (1H, dd, J=15.0, 4.1, Hₐ in SCH₂), 3.53 (1H, dd, J =15.0, 8.4, H_{b} in SCH₂), 2.68 (3H, s, SCH₃), 1.48 (3H, t, J=7.1, CH₃); ¹³C NMR (D₂O+CF₃COOD, 100 MHz); 169.64 (COONa), 168.63 (C₂), 151.11 (C₃ₐ), 147.22 (C₇), 141.26 (C₈), 51.55 (CHN), 50.29 (CH₂N), 29.30 (SCH₂), 13.16 (SCH₃), 12.23 (CH₃); IR (v/sm⁻¹): 1691 (C═O); Element, anal. calc. for C₁₀H₁₄N₆NaO₃S₂*H₂O, %: C—34.47, H—4.63, N—24.12, found: %: C —34.17, H—4.57, N—23.84.

Sodium salt of 2-methylthio-4-tert-butyl-6-(2′-amino-T-carboxyethylthio)-1,2,4-triazolo[5,1-c]1,2,4-triazine-7-one hydrate (17) was obtained from 9 using Procedure 5. The product is beige crystal powder, yield 27%, mp 168° C., ¹H NMR (D₂O, 400 MHz): 4.13 (1H, dd, J=9.4, 4.0, CHN), 3.98 (1H, dd, J=14.7, 4.0, Hₐ in SCH₂), 3.47 (1H, dd, J=14.7, 9.4, H_{b} in SCH₂), 2.70 (3H, s, SCH₃), 1.77 (9H, s, 3CH₃); ¹³C NMR (D₂O, 100 MHz): 164.29 (COONa), 151.27 (C₂), 149.26 (C₃ₐ), 146.80 (C₇), 135.60 (C₈), 67.89 (C(CH₃)₃), 64.08 (CHN), 28.12 (3CH₃), 27.51 (SCH₂), 13.79 (SCH₃); IR (v/sm⁻¹): 1696 (C═O); Element. anal. calc. for C₁₂H₁₇N₈NaO₃S₂*H₂O, %: C—36.17, H—4.81, N—21,09, found, %: C 36.29, H—4.78, N —21.12.

Sodium salt of 2-methylthio-4-pivaloyloxymethyl-6-(2+-amino-2′-carboxyethylthio)-1,2,4triazolo[5,1-c]1,2,4-triazine-7-one hydrate (18) was obtained from 10 using Procedure 5. The product is beige crystal powder, yield 35%, mp 163° C., ¹H NMR (D₂O, 400 MHz): 6.28 (2H, s, CH₂N), 4.21 (1H, dd, J=8.1, 4.2, CHN), 3.90 (1H, dd, J=15.0, 4,2, Hₐ in SCH₂), 3.55 (1H, dd, J =15.0, 8.1, H_{b} in SCH₂), 2.67 (3H, s, SCH₃), 1.18 (9H, s, 3CH₃); ¹³C NMR (D₂O+CF₃COOD, 100 MHz): 178.99 (C═OC(CH₃)₃), 169.33 (COONa), 168.88 (C₂), 151.22 (C₃ₐ). 146.62 (C₇), 143.23 (Cₐ, 74.67 (CH₂N), 51.65 (CHN), 38.62 (C(CH₃)), 29.37 (SCH₂), 25.89 (3CH₃), 13.21 (SCH₃); IR (v/sm⁻¹): 1711 (C═O); Element. anal. calc. for C₁₄H₁₉N₈NaO₅S₂*H₂O, %. C—36.84, H —4.64, N—18.41, found, %: C—36.53, H—4.51, N—18.48.

Procedure 6 for the synthesis of 2-methylsulfonyl-4-alkyl-6-(T-amino-2% carboxyethylthio)-1,2,4-triazolo[5,1-c]1,2,4-triazine-7-ones pentahydrates (19,20). 1,2,4-Triazolo-1,2,4-triazine (11 or 12) was dissolved in ethanol and added to a stirred fresh solution of cysteine hydrochloride (1 eq) in sodium bicarbonate (1 eq, 1 M). The reaction mixture was stirred for 1 h at room temperature, the solvent was removed in vacuo. The residue was recrystallized from ethanol (aq).

2-MethylsuIfonyl-4-methyl-6-(2′-amino-2′-carboxyethylthio)-1,2,4-triazolo[5,1-c]1,2,4-triazine-7-one pentahydrate (19) was obtained from 11 using Procedure 6. The product is yellow crystal powder, yield 56%, mp 102° C., ¹H NMR (D₂O, 400 MHz): 4.41 (1H, dd, J=6.4, 3.3, CHN), 3.51 (1H, dd, J=13.1, 3.3, Hₐ in SCH₂), 3.32 (1H, dd, J=13.1, 6.4, H_{b} in SCH₂), 3.28 (3H, s, SO₂CH₃), 3.25 (3H, s, NCH₃); ¹³C NMR (D₂O, 100 MHz): 174.74 (COOH), 166.76 (C₂), 160.00 (C₃ₐ,), 158.25 (C₈), 155.27 (C₇), 55.80 (CHN), 42.47 (SO₂CH₃), 40.23 (CH₃N), 29.88 (SCH₂); IR (v/sm⁻¹): 1615 (C═O); 1294, 1133 (—SO₂—); Element. anal. calc. for C₉H₁₂N₈O₅S₂*5H₂O, %: C—24.66, H—5.06, N—19.17, found, %: C—24.55, H—5.28, N—18.92.

2-Methylsulfonyl-4-ethyl-6-(2′-amino-2′-carboxyethylthio)-1,2,4-triazolo[5,1-c]1,2,4-triazine-7-one pentahydrate (20) was obtained from 12 using Procedure 6. The product is yellow crystal powder, yield 48%, mp 106° C., ¹H NMR (D₂O, 400 MHz): 4.36 (1H, dd, J=6.8, 3.3, CHN), 3.81-3.66 (2H, m, NCH₂), 3.43 (1H, dd, J=13.0, 3.3, H, in SCH₂), 3.28 (3H, s, SO₂CH₃), 3.24 (1H, dd, J=13.0, 6.8, H_{b} in SCH₂), 1.16 (3H, t, J=7.1, CH₃); ¹³C NMR (D₂O, 100 MHz): 174.72 (COOH), 164.17 (C₂), 160.06 (C₃ₐ), 158.22 (C₈), 152.46 (C₇), 55.84 (CHN), 50.02 (NCH₂), 42.47 (SO₂CH₃), 30.04 (SCH₂), 11.72 (CH₃); IR (v/sm⁻¹): 1613 (C═O); 1287, 1130 (—SO₂—); Element. anal. calc. for C₁₀H₁₄N₈S₂*5H₂O, %: C 26.55, H—5.35, N—18.57, found, %: C—26.65, H—5.38, N—18.45.

### Pharmaceutical Compositions

The invention also provides a pharmaceutical composition comprising the arginine or cysteine salt of Formula 1, or one of the compounds in Table 2, or the arginine salt of one of the compounds in Table 2, or the cysteine salt of one of the compounds in Table 2, or any combination thereof, and a pharmaceutically acceptable carrier.

The pharmaceutical composition may further comprise or be administered in combination with one or more other antiviral agents including, but not limited to, oseltamivir phosphate, zanamivir or Virazole®.

The term ‘composition’ is intended to include the formulation of an active ingredient with conventional carriers and excipients, and also with encapsulating materials as the carrier, to give a capsule in which the active ingredient (with or without other carriers) is surrounded by the encapsulation carrier. Any carrier must be ‘pharmaceutically acceptable’ meaning that it is compatible with the other ingredients of the composition and is not deleterious to a subject. The compositions of the present invention may contain other therapeutic agents as described above, and may be formulated, for example, by employing conventional solid or liquid vehicles or diluents, as well as pharmaceutical additives of a type appropriate to the mode of desired administration (for example, excipients, binders, preservatives, stabilizers, flavors and the like) according to techniques such as those well known in the art of pharmaceutical formulation (see, for example, Remington: The Science and Practice of Pharmacy, 21st Ed., 2005, Lippincott Williams & Wilkins).

The pharmaceutical composition includes those suitable for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances that may also act as diluents, flavoring agents, solubilizers, lubricants, suspending agents, binders, preservatives, tablet disintegrating agents, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term ‘preparation’ is intended to include the formulation of the active compound with an encapsulating material as the carrier by providing a capsule in which the active component, with or without carriers, is surrounded by a carrier, which is thus in association with it. Similarly. cachets and lozenges are included. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The compositions according to the present invention may thus be formulated for parenteral administration (for example, by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, for example, sterile, pyrogen -free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for the compounds, and may contain, for example, water, ethanol, polyol (for example, glycerol, propylene glycol and liquid polyethylene glycol and the like), suitable mixtures thereof, and vegetable oils.

Pharmaceutical forms suitable for injection may be delivered by any appropriate route including intravenous, intramuscular, intracerebral, intrathecal, epidural injection or infusion. Sterile injectable solutions are prepared by incorporating the active compounds in the required amount in the appropriate solvent with various other ingredients such as these enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

When the active ingredients are suitably protected they may be orally administered, for example, with an inert diluent or with an edible carrier capable of assimilation, or they may be enclosed in hard or soft shell gelatin capsule, or they may be compressed into tablets, or they may be incorporated directly with the food of the diet. For oral therapeutic administration, the active compound may be incorporated with excipients and used in the form of ingestible tablets, buccal tablets, troches, capsules, elixirs, suspensions, syrups, wafers and the like.

The amount of active compound in therapeutically useful compositions should be sufficient hat a suitable dosage will be obtained.

The tablets, troches, pills, capsules and the like may also contain the components as listed hereafter; a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin; or a flavoring agent such as peppermint, oil of wintergreen, or cherry flavoring. When the dosage unit form is a capsule, it may contain, in addition to materials of the above type, a liquid carrier.

Various other materials may be present as coatings or to otherwise modify the physical form of the dosage unit, For instance_{;} tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active ingredient to specific regions of the gut.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. To improve nasal delivery and retention the compounds according to the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, a hydrofluorocarbon (HFC) for example hydrofluoroalkanes (HFA), carbon dioxide, or other suitable gas.

The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, for example gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Dosage unit form as used herein refers to physically discrete units suited as unitary dosages for the subjects to be treated, each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of viral infection in living subjects having a diseased condition in which bodily health is impaired as herein disclosed in detail.

The invention also includes the compounds in the absence of carrier where the compounds are in unit dosage form.

Liquids or powders for intranasal administration, tablets or capsules for oral administration and liquids for intravenous administration are the preferred compositions.

### Methods of Treatment

The compounds of the invention have demonstrated potency as antiviral agents and therefore offer a method of treating a viral infection. The compounds of the invention can also be used to treat a viral disease or reduce exacerbation of an underlying or pre-existing respiratory disease wherein a viral infection is a cause of said exacerbation. The viral disease may include brochiolitis or pneumonia. The underlying or pre-existing respiratory diseases or conditions may include asthma, chronic obstructive pulmonary disease (COPD) and immunosuppression such as immunosuppression experienced by bone marrow transplant recipients.

Treatment may be therapeutic treatment or prophylactic treatment. Generally, the term ‘treating’ means affecting a subject, tissue or cell to obtain a desired pharmacological and/or physiological effect and includes: (a) inhibiting the viral infection or viral disease, such as by arresting its development or further development; (b) relieving or ameliorating the effects of the viral infection or viral disease, such as by causing regression of the effects of the viral infection or viral disease; (c) reducing the incidence of the viral infection or viral disease or (d) preventing the viral infection or viral disease from occurring in a subject, tissue or cell predisposed to the viral infection or viral disease or at risk thereof, but has not yet been diagnosed with a protective pharmacological and/or physiological effect so that the viral infection or viral disease does not develop or occur in the subject, tissue or cell.

The term ‘subject’ refers to any animal, in particular mammals such as humans, having a disease that requires treatment with the compounds of the invention. Particularly preferred treatment groups include at risk populations such as hospitalized subjects. the elderly, high-risk adults and infants. The compounds of the invention may also be used in agriculture and to treat farm animals including, but not limited to, chickens, ducks or pigs.

The term ‘administering’ should be understood to mean providing a compound or pharmaceutical composition of the invention to a subject suffering from or at risk of the disease or condition to be treated or prevented.

Although the invention has been described with reference to treating influenza, RSV and PIV infections and diseases, it will be appreciated that the invention may also be useful in the treatment of other viruses including, but not limited to, Rift Valley fever virus, West Equine Encephalomyelitis (WEE) viruses, parainfluenza, respiratory-syncytium virus (RSV), Aujeszky's disease virus, avian infectious laryngotracheitis virus, West Nile Virus, virus tick-borne encephalitis, SARS and avian influenza.

### Dosages

The term ‘therapeutically effective amount’ refers to the amount of the compound of the invention that will elicit the biological or medical response of a subject, tissue or cell that is being sought by the researcher, veterinarian, medical doctor or other clinician.

In the treatment of viral infections or diseases, an appropriate dosage level will generally be about 0.01 to about 500 mg per kg subject body weight per day which can be administered in single or multiple doses. The dosage may be selected, for example, to any dose within any of these ranges, for therapeutic efficacy and/or symptomatic adjustment of the dosage to the subject to be treated.

It will be understood that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the subject undergoing therapy.

What is claimed is:

1. A compound selected from the group consisting of compound 1a, compound 3a, compound 3b, compound 3c, compound 3d, compound 4a, compound 4b, compound 4c, compound 4d, compound 5, compound 6, compound 7, compound 8, compound 9, compound 10, compound 11, compound 12, compound 13, compound 14, compound 15, compound 16, compound 17, compound 18, compound 19 and compound 20.

2. The molecule of compound 1a as an antiviral agent.

3. The molecule of compound 3a as an antiviral agent.

4. The molecule of compound 3b as an antiviral agent.

5. The molecule of compound 3c as an antiviral agent.

6. The molecule of compound 3d as an antiviral agent.

7. The molecule of compound 4b as an antiviral agent.

8. The molecule of compound 4d as an antiviral agent.

9. The molecule of compound 5 as an antiviral agent.

10. The molecule of compound 6 as an antiviral agent.

11. The molecule of compound 7 as an antiviral agent.

12. The molecule of compound 8 as an antiviral agent.

13. The molecule of compound 9 as an antiviral agent.

14. The molecule of compound 10 as an antiviral agent.

15. The molecule of compound 12 as an antiviral agent.

16. The molecule of compound 13 as an antiviral agent.

17. The molecule of compound 14 as an antiviral agent.

18. The molecule of compound 18 as an antiviral agent.

19. A compound having antiviral activity, comprising a salt selected from the list of the 1a, the arginine salt of compound 3a, the arginine salt of compound 3b, the arginine salt of compound 3c, the arginine salt of compound 3d, the arginine salt of compound 4b, the arginine salt of compound 4d, the arginine salt of compound 5, the arginine salt of compound 6, the arginine salt of compound 7, the arginine salt of compound 8, the arginine salt of compound 9, the arginine salt of compound 10, the arginine salt of compound 12, the arginine salt of compound 13, the arginine salt of compound 14, the arginine salt of compound 18, the cysteine salt of compound 1a, the cysteine salt of compound 3a, the cysteine salt of compound 3b, the cysteine salt of compound 3c, the cysteine salt of compound 3d, the cysteine salt of compound 4b, the cysteine salt of compound 4d, the cysteine salt of compound 5, the cysteine salt of compound 6, the cysteine salt of compound 7, the cysteine salt of compound 8, the cysteine salt of compound 9, the cysteine salt of compound 10, the cysteine salt of compound 12, the cysteine salt of compound 13, the cysteine salt of compound 14 and the cysteine salt of compound 18.

20. A compound according to any of claims 1-19 for use in a therapeutic or prophylactic treatment.

21. A compound according to any of claims 1-20 for use in the treatment or prophylaxis of an infectious disease.

22. Compound according to any of claims 1-20 for use in the treatment or prophylaxis of an infectious disease in combination with an additional anti-infective agent,

23. Use of a compound as defined in any of the preceding claims 1-22 for the manufacture of a medicament for the treatment or prophylaxis of an infectious disease.

24. Use of a compound as defined in any of the preceding claims 1-22, wherein said compound is used or administered in a clinically relevant amount.

25. A pharmaceutical composition comprising a compound according to any of claims 1-19 or salts, racemates, isomers or prodrugs thereof and a pharmaceutically acceptable carrier or excipient.

26. A pharmaceutical composition according to claim 20, comprising a clinically relevant amount of the compound according to any of claims 1-25.

27. The compounds and compositions of claims 1-26 to treat a disease caused by a virus selected from the group consisting of Influenza A (including, but not limited to, all varieties of HxNy e.g H1N1, H3N2, H5N1), Influenza B, West Nile Virus, Dengue, members of the flaviviridae family including, but not limited to, Yellow Fever Virus and members of the Alphaviridae family including, but not limited to, Rift Valley Fever and Venezuelan Equine encephalitis, coronaviruses including, but not limited to, SARS, virus tick-borne encephalitis, respiratory syncytial virus, herpes simplex virus and pox viruses.

28. The methods of manufacturing the compounds of claims 1-19 as described above.
